(19) United States
(12) Patent Application Publication
(10) Pub. No.: US 2015/0293102 A1
(43) Pub. Date: Oct. 15, 2015

(54) Detecting low-abundant analyte in microfluidic droplets

(71) Applicant: Jung-uk Shim, Cambridge (GB)

(72) Inventor: Jung-uk Shim, Cambridge (GB)

(21) Appl. No.: 14/249,373

(22) Filed: Apr. 10, 2014

(51) Int. Cl. G01N 33/574; G01N 33/543

(52) U.S. Cl. CPC G01N 33/57434; G01N 33/54393; G01N 2333/96441

(57) ABSTRACT

A method to produce aqueous droplets in oil and to manipulate the droplets for storage in the microfluidic device for certain amount of time to accumulate detectable amount of product produced by a single copy or plural copies of enzyme enclosed in the droplets, and to detect and measure the biomarkers in the antibody binding assay is disclosed. The method comprises: (1) generation of droplets in the microfluidic device, (2) storage of droplets in the microfluidic device, (3) measurement of activity of a single copy or plural copies of enzyme in the droplets, (4) individual molecule-counting immunoassay using the droplets.

Applications can include the single molecule counting immunoassay, a platform for extremely high through digital PCR, a platform for directed evolution at individual molecule resolutions, nanoparticles synthesis, biodegradable polymer particle production and single molecule analysis.

### CROSS-REFERENCE TO RELATED APPLICATIONS

This application claims benefit of U.S. provisional patent application No. 61/811,709 filed on Apr. 13, 2013, priority to U.K. patent application No. GB1207031.4 filed on Apr. 23, 2012.

### FIELD OF THE INVENTION

The present invention relates to systems and methods for detecting analyte molecules or particles in a fluid sample and in some cases, determining a measure of the concentration of the molecules or particles in the fluid sample. Methods of the present invention may comprise immobilizing a plurality of analyte molecules or particles with respect to a plurality of capture particles. At least a portion of the plurality of capture particles may be spatially separated into a plurality of locations. A measure of the concentration of analyte molecules in a fluid sample may be determined, at least in part, on the number of reaction vessels comprising an analyte molecule immobilized with respect to a capture particle. In some cases, the assay may additionally comprise steps including binding ligands, precursor labelling agents, and/or enzymatic components.

This invention relates to methods for microfluidic generation and storage of droplets, for fabrication of microfluidic devices. Embodiments of the methods are particularly useful for single-molecule counting immunoassay and polymer particle synthesis.

### BACKGROUND OF THE INVENTION

Water-in-oil droplets are emerging as a potentially powerful technology to quantitatively study compartmentalized reactions of single enzyme molecules or single cells because the concentration of reaction products or secreted molecules exceed the detection threshold much more rapidly in small confined volumes than in bulk solution. In order for the enzymatic product to be detectable using epifluorescence microscopy, the volume of the reaction chamber containing the enzyme and its fluorogenic substrate have been reduced to less than 100 femtoliter. In this volume, a single molecule of enzyme has a concentration of ˜17 picomolar, enabling substrate turnover to dominate processes such as uncatalyzed hydrolysis, which in turn allows rapid accumulation and detection of the product. Due to their inherent scalability, droplet-based platforms could enable numerous single-molecule assays to be performed in parallel.

According to literature written by Rotman et al [B. Rotman, Proc. Natl. Acad. Sci. U.S.A. 1961, 47, 1981] and Lee et al [A. I. Lee, J. P. Brody, Biophys. J. 2005, 88, 4303], ultra-small droplet with volumes ranging from 0.5 fL to 2 pL have been used to detect the activity of single enzyme molecules, but the polydispersity of the emulsions used limited the precision and throughput of these studies.

According to literature written by Theberge et al [A. B. Theberge, F. Courtois, Y. Schaerli, M. Fischlechner, C. Abell, F. Hollfelder, W. T. S. Huck, Angew. Chem., Int. Ed. Engl. 2009, 49, 5846], Chiu et al [D. T. Chiu, R. M. Lorenz, G. D. M. Jeffries, Anal. Chem. 2009, 81, 5111], Guo et al [M. T. Guo, A. Rotem, J. A. Heyman, D. A. Weitz, Lab Chip 2012], there has been tremendous progress in the development of microfluidics-based droplet platforms for the on-chip formation and manipulation of monodisperse droplets, and the associated use of a range of fluorescence-based techniques for high-throughput and highly sensitive analysis of droplet contents. Existing microfluidic devices generate highly monodisperse droplets at the pico- to nanoliter scale. In such volumes, according to literature written by Joensson et al [H. N. Joensson, M. L. Samuels, E. R. Brouzes, M. Medkova, M. Uhlen, D. R. Link, H. Andersson-Svahn, Angew. Chem., Int. Ed. Engl. 2009, 48, 2518.], several hours of enzymatic activity are required to turn over sufficient substrate for single enzyme molecule detection. Furthermore, maximal droplet generation rates are in the 10 kHz range, limiting high-throughput measurements of fast reactions.

The gold standard immunoassay, ELISA (enzyme-linked immunosorbent assay), enables the detection of biomarkers at concentrations above picomolar (10⁻¹² M), but there remains an unmet clinical need for detection of biomarkers of neurodegenerative diseases and cancers that are present in biological fluids at concentrations in the range of 10⁻¹²-10⁻¹⁶ M; the ability to detect single enzyme molecules provides a means to quantitate such low abundance markers.

According to literature written by Rissin et al [D. M. Rissin, C. W. Kan, T. G. Campbell, S. C. Howes, D. R. Fournier, L. Song, T. Piech, P. P. Patel, L. Chang, A. J. Rivnak, E. P. Ferrell, J. D. Randall, G. K. Provuncher, D. R. Walt, D. C. Duffy, Nat. Biotechnol. 2010, 28, 595.], Zhang et al [H. B. Zhang, S. Nie, C. M. Etson, R. M. Wang, D. R. Walt, Lab Chip 2012, 12, 2229.], Kan et al [C. W. Kan, A. J. Rivnak, T. G. Campbell, T. Piech, D. M. Rissin, M. Mosl, A. Peterca, H. P. Niederberger, K. A. Minnehan, P. P. Patel, E. P. Ferrell, R. E. Meyer, L. Chang, D. H. Wilson, D. R. Fournier, D. C. Duffy, Lab Chip 2012, 12, 977.] and Kim et al [S. H. Kim, S. Iwai, S. Araki, S. Sakakihara, R. lino, H. Noji, Lab Chip 2012.], one promising approach uses the turnover of a fluorogenic substrate by single enzyme molecules within well-arrays as the basis for ultra sensitive digital ELISA.

However, the need for mechanical fabrication of these femtoliter reaction chambers places inherent limits on the scalability and flexibility of ultra sensitive diagnostic assays, which could be overcome using a droplet-based approach.

### BRIEF SUMMARY OF INVENTION

According to the present invention there is therefore provided a method of fabricating a multilayered microfluidic device that enables the generation and on-chip manipulation of highly monodisperse femtoliter droplets at frequencies up to a few mega-hertz. This innovation allows the measurement of enzymatic activity of single enzyme molecules in a few minutes, a property that have been exploited to construct a bead-based ELISA for the detection of a low-abundance protein biomarker.

I invented a flow focusing nozzle having locally shallower depth and width to obtain a substantial enhancement of flow speed without a significant increase of the internal pressure. The local constriction is introduced within a section of the device, where the channel dimensions are reduced (FIG. 1, FIG. 2).

I invented a microfluidic component for storing femtodroplets for a sufficient time to monitor chemical reactions therein. A wide and shallow storage area is integrated in the microfluidic device to trap and keep femtodroplets for long duration of time enough to accumulate certain amount of products. The storage area is divided into a few tens or hundreds of traps, each of which is isolated by monolithic microfluidic valves (FIG. 2, FIG. 3, FIG. 4, FIG. 7).

I invented a method to measure the enzymatic activity of individual enzyme molecules using the femtodroplets in the microfluidic device. The enzymatic activity of individual molecules can be interrogated in femtodroplets. As the enzymatic turn-over starts at the droplet generation, the initiation of chemical reaction in stored femtodroplets is perfectly synchronized, and thus can be precisely monitored in time. The time course fluorescence of femtodroplets stored in each trap is imaged in order to yield kinetic information of the chemical reactions in each droplet (FIG. 7, FIG. 8, FIG. 9, FIG. 10, FIG. 11, FIG. 12).

I invented a digital immunoassay using the femtodroplet assay and a bead-based antibody binding assay, termed the femtodroplet immunoassay, which is able to quantify very low concentration of biomarkers. I exploited the ability of the femtodroplet assay to detect the presence of single enzymes in order to measure concentrations of target analyte which is conjugated with enzyme reporters (FIG. 13, FIG. 15, FIG. 16, FIG. 17).

I invented a method to perform identical repetitive femtodroplet immunoassay in a single assay. The embedded microfluidic valve is conveniently controlled to flush stored femtodroplets out of and reload freshly generated femtodroplets into those traps by application and release of external pressure. This is done in seconds due to the extremely frequent droplet generation so that it enables us to conduct identical repetitive assays in every a few minutes for demanded time duration (FIG. 14).

I invented a method to identify presence of beads using fluorescence of protein. I found that the capture-antibody conjugated beads are fluorescent due to the intrinsic fluorescence of immunoglobin. The bead fluorescence is strong enough to be observable in red-fluorescence and at a same time weak enough for single enzyme activity in the femtodroplet to be differentiated in green-fluorescence so that it enables us to count the number of beads more accurately and comfortably than when using the bright field images (FIG. 16).

I invented a method to enhance the detection throughput of the femtodroplet immunoassay by encapsulation of multiple beads in a droplet. In order to encapsulate one bead per droplet only 10% of droplets are occupied by beads and the rest, 90%, have no bead. To get rid of this inefficiency of droplet usage multiple beads in a droplet can be encapsulated. Encapsulation of multiple beads maximizes the usage of droplets, thus reduces the time to detect the target molecule and speeds up the throughput; therefore it enhances the sensitivity.

### BRIEF DESCRIPTION OF DRAWINGS

FIG. 1 Femtodroplet formation at the nozzle of the microfluidic device. Droplets with a typical volume of 32 fL are generated at a frequency of around 3.5×10⁵ per second.

FIG. 2 Photograph of the whole multilayered PDMS device. The upper layer consists of the nozzle (10 μm wide×5 μm deep), flow channels (100 μm wide×25 μm deep) and storage compartments (2 mm wide×7 mm long×5 μm deep), with a capacity for ˜2×10⁵ femtodroplets. The bottom layer houses the monolithic valves used to control droplet flow and isolate the traps. There are injection holes for introduction of fluids into the device; the outer two for oil and the others for aqueous solutions. When the main valve is closed, the stream of femtodroplets is directed into the storage region (stream path 1). If this valve is opened, droplets flow out of the device by stream path 2 due to the lower flow resistance encountered.

FIG. 3 Image of traps used for femtodroplet storage and isolation. The contents of the traps are manipulated by the action of networks of embedded monolithic valves in response to external pressure.

FIG. 4 Vertical schematic of the storage structure. When pressure is applied, the thin PDMS membrane (15 μm thick) bends up to seal off the flow and traps the femtodroplets.

FIG. 5 Generation frequency and volume of femtodroplets as a function of the oil flow rates (Qₒᵢₗ) at a constant water flow rate (Q_{water}) of 40 μL/hr. In order to generate 32 fL droplets at a frequency of 350 kHz, 230 μL/hr of oil and 40 μL/hr of water in flow rates were introduced. The dashed line is a prediction curve, fitting the experimental data. The linearity (r-square) is 0.98.

FIG. 6 The histogram of the droplet volume. The volume shown is 35.5 fL and the standard deviation is 0.66 fL at 40 μL/hr of water and 220 μL/hr of oil.

FIG. 7 Femtodroplets stored in a trap. The supporting posts maintain the shallow trap structure (5 μm deep). Droplets were packed in a monolayer in a trap (right).

FIG. 8 Images showing green fluorescence resulting from hydrolysis of FDG (250 μM) by β-galactosidase (1.5×10⁻³ unit/ml, equivalent to 2.1 pM) in femtodroplets after 1 and 10 min. The bright spots represent femtodroplets enclosing a single enzyme molecule, in which fluorescein reaction product is generated.

FIG. 9 Representative time traces of enzyme activity measured in femtodroplets that contain either one β-galactosidase molecule or none. The fluorescence was measured every minute and converted to concentrations of fluorescein using a calibration curve after correcting for photobleaching. The black dashed line represents a threshold, defined above three standard deviations of the background at 10 minute. The positive traces show a range of activities.

FIG. 10 Product concentration increase per single copy of β-galactosidase as a function of substrate (500, 250, 125, 63, 25, 13 μM FDG). Taking the time derivative of the fluorescein concentration produced in the enzymatically-active droplets in FIG. 9 yields the concentration increase. The error bar is the standard deviation. The data can be fit to the Michaelis-Menten equation, υ=Vₘₐₓ·[S]/(Kₘ+[S]), giving a good correlation (r²=0.97) and a Kₘ of 90±26 μM.

FIG. 11 Fluorescence micrographs of traps after 10 min incubation at various enzyme concentrations. The fraction of stored femtodroplets that show product formation varies in a concentration-dependent manner.

FIG. 12 Plot of the prepared concentration (where one unit of enzyme hydrolyzes 1 μM of o-nitrophenyl β-D-galactoside to o-nitrophenol and D-galactose per minute at pH 7.3 at 37° C.) vs. the experimentally-determined molar concentration of β-galactosidase. The dotted line represents a linear fit.

FIG. 13. Binding of an antigen to antibody-coated beads; a single bead-captured target molecule is subsequently sandwiched by a biotinylated detection-antibody and a streptavidin-β-galactosidase conjugate.

FIG. 14 Beads with or without an immunocomplex are singly encapsulated in femtodroplets with a substrate (FDG) and subsequently in-line incubated in the trap to accumulate the fluorescent product of single enzyme reporter. Each trap can enclose about 5×10³ droplets so the capacity of the current storage is about 2×10⁵ droplets. The stored droplets can be completely flushed out and reloaded in 10 seconds due to the extremely high-speed generation of droplets.

FIG. 15 After the in-line incubation, three populations of femtodroplets are observed, i) droplets containing no bead ii) those containing a bead without immunocomplexes and iii) those containing a bead with an immunocomplex exhibiting a positive fluorescence signal due to the enzymatic activity of single enzyme reporter. The numerical ratio of (iii) to ((ii)+(iii)) yields the concentration of the target molecules. Thus, the larger the number of available droplets in a measurement is, the lower the detection sensitivity can be accomplished in a given assay time.

FIG. 16 Brightfield, red- and green-fluorescence images (left to right) of stored femtodroplets containing anti-PSA coated beads (10 pM) and substrate (FDG, 250 μM) after a 10-minute incubation period following immunoassay (in the presence of a 140 pg/mL concentration of PSA) and subsequent encapsulation. Bright spots in the red-fluorescence micrograph result from beads conjugated with the capture-antibody, while those in the green-fluorescence image are due to femtodroplets in which enzymatic activity has occurred. The green circles in all images represent droplets that contain a bead and show enzymatic activity, while the red circles in the bottom left corner of each panel indicate a single femtodroplet that exhibits enzymatic activity but which does not contain a bead, indicating the presence of unbound enzyme in the droplet.

FIG. 17 Plot of the molarity of PSA measured by the droplet-based immunoassay vs. the prepared concentration. Molar concentrations were calculated from a Poisson distribution function as described in FIG. 12, except that the fraction of beads encapsulated in droplets showing enzymatic activity (e.g. the number ratio of green circles to fluorescent beads in FIG. 16) and the known bead concentration (10 pM) were used instead of the inactive fraction and volume of the femotodroplets. The dotted line represents a linear fit.

### DETAILED DESCRIPTION OF THE INVENTION

I describe a microfluidic device that is able to generate and manipulate droplets with volumes of 1-100 fL at MHz frequencies. This femtoliter microfluidic droplet-based approach enables the measurement of the activity of a single copy of an enzyme and can be exploited to quantify very low-abundance biomarkers by integrating a bead-based immunoassay with direct counting of individual enzyme molecules for creating a highly sensitive diagnostic test. The fluidic femtodroplet reaction chambers used in this study offer significant advantages due to the robustness and flexibility of the microfluidic circuit compare to the digital ELISAs reported by Rissin et al [Nat. Biotechnol. 28, 595-U525 (2010)]: extremely high-speed generation and manipulation of fast-flowing droplets, the ability to carry out replicate assays without replacing hardware enabling a significant enhancement of the sampling size, ease of automation and integration with other fluidic sample preparation modules and the possibility of varying the size of the reactors at will.

### 1. Generation and Manipulation of Femtoliters Volume Microfluidic Droplets

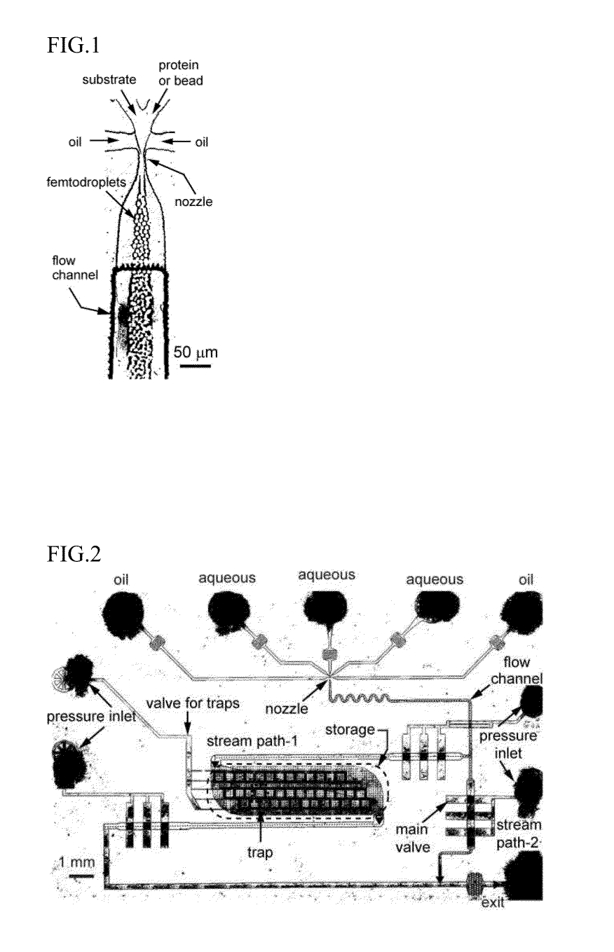

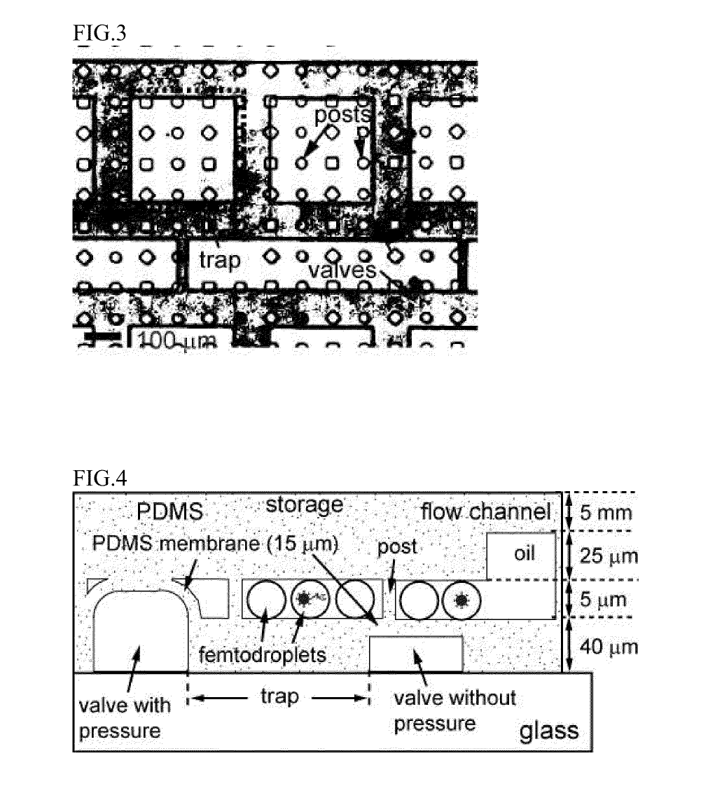

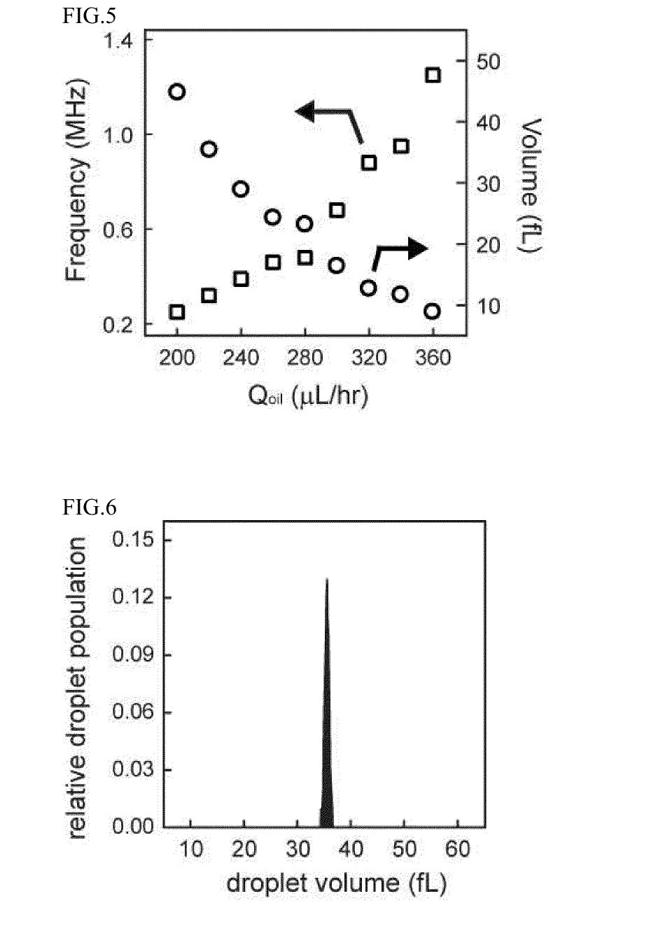

I invented a microfluidic device that is able to generate controllably and manipulate water droplets in oil of 1-100 femtoliter volume—which I call femtodroplets—at frequencies>1 MHz (FIG. 1, FIG. 5). Microfluidic droplets can be generated by shearing one fluid (water) by a second immiscible one (oil). In order to produce small water droplets at high frequencies, a large shear force and low interfacial tension at the oil-water interface are required according to Yobas et al [Yobas, L., Martens, S., Ong, W. L. Ranganathan, N, Lab Chip 6, 1073-1079 (2006)]. Large shear forces can be generated by either applying a higher flow rate of oil or reducing the channel dimensions in order to increase the flow speed. However, high flow rates can lead to difficulties in device operation and smaller channel dimensions produce high internal pressure, inversely proportional to the fourth power of the channel diameter according to Beebe et al [Beebe, D. J., Mensing, G. A. & Walker, G. M. Annu. Rev. Biomed. Eng. 4, 261-286 (2002)]. In order to substantially enhance the flow speed during droplet formation without generating high internal pressure throughout the flow channel in the device, a flow-focusing nozzle was integrated into the design of my device. This strategy introduces a local constriction within a local section of the device, for example, 300-μm, where the channel dimensions are reduced, for example, from 100×25 μm (width×depth) to 10×5 μm (FIG. 1, FIG. 2). This nozzle enables the controlled generation of highly monodisperse aqueous droplets in oil, for example, fluorinated oil (HFE-7500, Novec™, 3M), previously mixed with a surfactant, for example 5% w/w, at frequencies of 10⁵-10⁶ Hz (FIG. 5, FIG. 6). The interfacial tension (IFT) between the oil and water exhibited by this mixture is extremely low, for example ˜3 mN/m, which allows the generation of small droplets at much higher frequencies than is possible with other oils, e.g. silicone oil (IFT˜38 mN/m) and mineral oil (IFT˜51 mN/m). The frequency of droplet-formation was measured using a confocal optical setup, and the droplet volume calculated from the formation frequency and the flow rate of water. Using the current experimental setup, the frequency is maximally measurable up to 1.3 MHz, leading to a femtodroplet volume of 8.6 fL. However, very stable droplet generation at an oil flow rate of 480 μL/hr was observed, where the droplet-generation frequency is expected to be 3.1 MHz according to the curve fit, implying a femtodroplet volume of 3.6 fL. This droplet generation frequency is about two orders of magnitude faster than previously reported according to Theberge, A. B. et al. [Angew. Chem., Int. Ed. Engl. 49, 5846-5868 (2009)]. I conclude that the low interfacial tension, for example less than 10 mN/m and the locally narrow flow-focusing nozzle design, for example a local section of less than 10 μm depth and about 300 μm long, are key features enabling controllable generations of femtoliter droplets at millions-hertz frequencies. The femtodroplets formed using my device provide discrete reaction compartments that are small enough to enable the products of one molecule of enzyme to be detected within minutes by epifluorescence microscopy but also large enough to be manipulated fluidically.

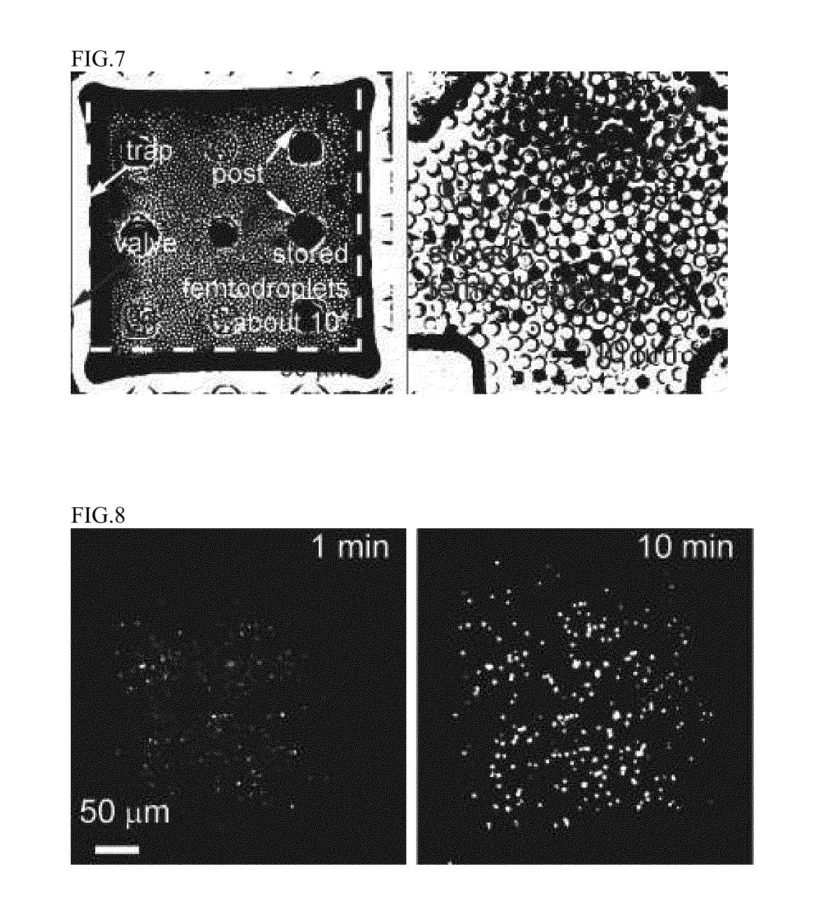

Once single enzyme molecules and the fluorogenic substrate have been encapsulated, it takes a few minutes to accumulate a measurable amount of fluorescent product. A storage area, for example 2 mm×7 mm×5 μm (length×width×depth), was therefore integrated into the microfluidic device to store femtodroplets while the enzymatic reaction occurs (FIG. 2). The storage area is divided into a number of traps, for example 40 traps with for example 300 μm×300 μm wide, isolated by monolithic microfluidic valves (FIG. 2, FIG. 3, FIG. 4). As the depth, for example 5 μm, of the storage area is comparable to the diameter of the femtodroplets, droplets stored in the microfluidic device are packed into a monolayer that allows fluorescence measurements of individual droplets using a simple epifluorescence microscope (FIG. 7). Trapping the femtodroplets in this way allows enzymatic activity of specific enzymes to be monitored continuously inside thousands of droplets simultaneously (FIG. 8). An embedded microfluidic valve is used to flush stored droplets out of the traps and reload freshly-generated femtodroplets by application and release of external pressure, for example about 50 psi. This process takes only about 10 seconds due to the extremely high frequency of droplet generation and it is therefore not rate-limiting for assay repetition.

### 2. Measurement of Enzymatic Reaction of Individual Enzyme Molecules

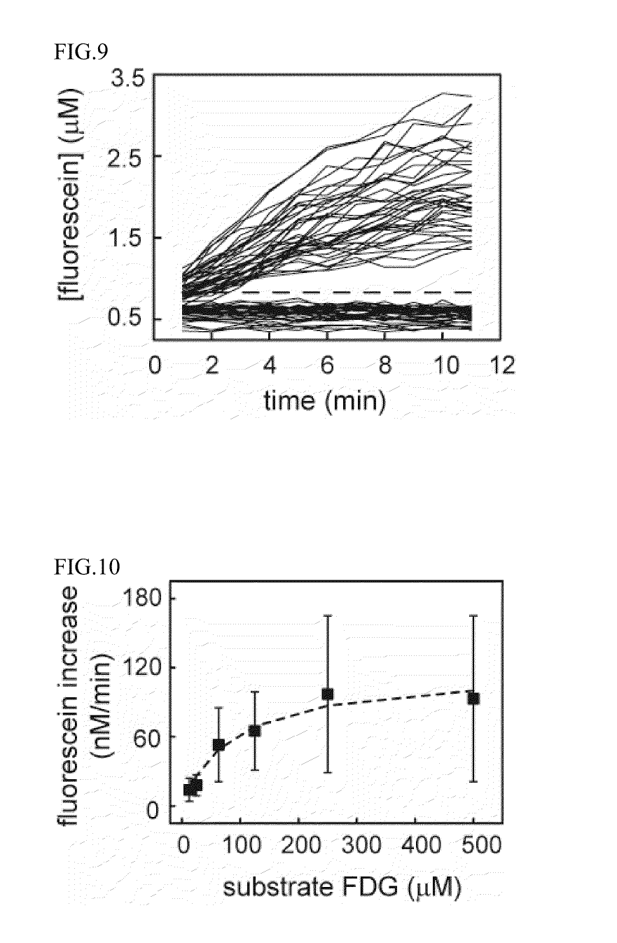

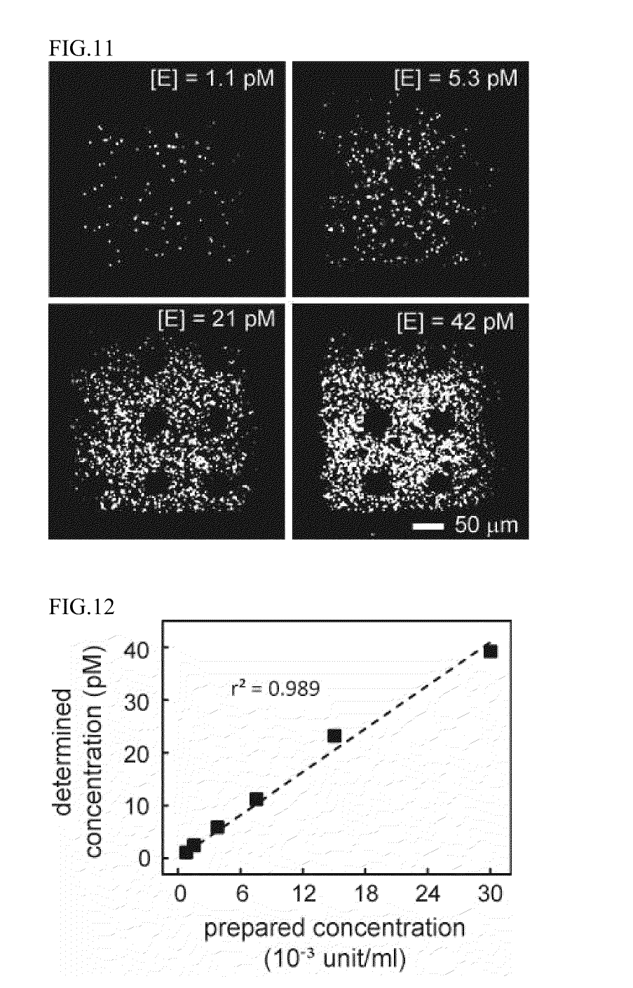

I first determined the time required for individual molecules of β-galactosidase encapsulated in 32 fL droplets to generate sufficient fluorescence signal to be detectable above the background from 250 μM of a substrate (fluorescein-di-β-D-galactopyranoside, FDG). As enzymatic turnover starts at droplet generation, the initiation of the chemical reaction in each femtodroplet occurs within a second of each other, and so can be precisely monitored temporally. The time course of fluorescence generation in approximately 5×10³ femtodroplets stored in each trap was imaged at enzyme concentrations of up to 3×10⁻² unit/mL (equivalent to about 40 pM) where likelihood of enzyme occupancy of each droplet is <0.8 (FIG. 11). After incubation for 10 minutes, two populations of droplets were clearly visible (FIG. 8). The fraction of bright femtodroplets (FIG. 11)—with intensities separated from the mean fluorescence of the other dark droplet population by >3 s.d. (FIG. 9)—followed a Poisson distribution as a function of prepared enzyme concentration, as expected if the observed product formation is due to the activity of single molecules of β-galactosidase. The fraction of enzymatically-inactive femtodroplets (i.e. n=0) was inserted into a Poisson distribution function, ƒ(n)=λⁿ·e^{-λ}/n!, where n describes the number of enzyme molecules in a droplet, yielding the average occupancy per droplet. The molar concentration of enzyme was then calculated from the average occupancy and the femtodroplet volume (32 fL). The linear relation between the prepared concentration and the determined concentration of β-galactosidase in FIG. 12 confirmed that the enzymatic activity observed in the bright femtodroplets is due to single enzyme molecules.

The enzymatic activity of individual molecules of β-galactosidase (3.8×10⁻³ unit/mL, equivalent to about 5 pM) was also kinetically-characterized in femtodroplets at various substrate concentrations with each experiment monitoring more than 150 enzyme molecules stored in each trap (FIG. 9, FIG. 10). The lowest substrate concentration was 13 μM, so enough substrate is present to eliminate the effect of substrate depletion. The averaged enzymatic activity of individual enzyme molecules depends asymptotically on substrate concentrations according to the Michaelis-Menten equation (FIG. 10). The averaged Michaelis constant (Km) of femtodroplet-encapsulated β-galactosidase was 90 μM, which closely matched that measured in bulk (124 μM). However, single-molecule measurement of enzyme kinetics revealed significant molecule-to-molecule variation in activity: the coefficients of variation (ratio of the standard deviation to the mean) are 0.64 and 0.13 for single enzyme and ensemble measurements, respectively. This wide distribution likely reflects the existence of considerable variation of activities within a population of enzyme, which has also been reported by other laboratories.

### 3. Detection of a Cancer Biomarker Using a Femtodroplet Assay

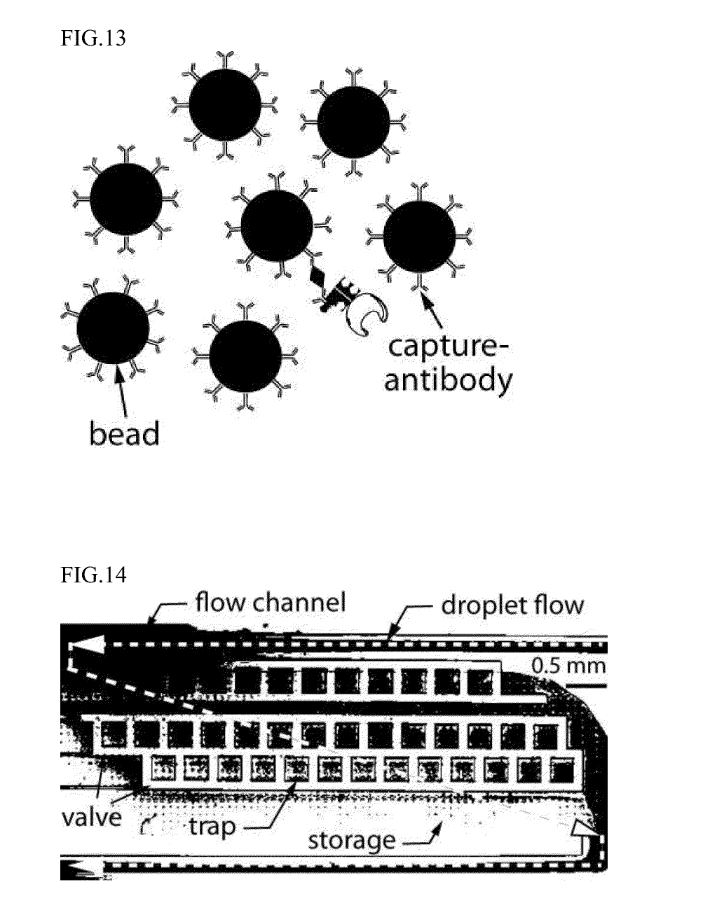

The ability to sensitively detect β-galactosidase, a typical reporter enzyme, paves the way for ultrasensitive diagnostics using a bead-based ELISA to quantify very low concentrations of the biomarker prostate-specific antigen (PSA) reported by a single enzyme. A monoclonal antibody to the target protein was covalently coupled to polystyrene beads, for example 1 μm diameter, to enable capture in PBS buffer and subsequent detection of PSA in a sandwich complex containing a detector antibody specifically bound to a β-galactosidase reporter (FIG. 13). The capture antibody-functionalised beads exhibited red autofluorescence, possibly due to the intrinsic fluorescence of immunoglobin according to Eftink, M. R. [Methods Biochem. Anal. 35, 127-205 (1991)]. This made it possible to count the number of beads by fluorescence imaging more easily than by using brightfield illumination, without interfering with the detection of enzymatically-produced fluorescein in the green part of the spectrum (FIG. 16).

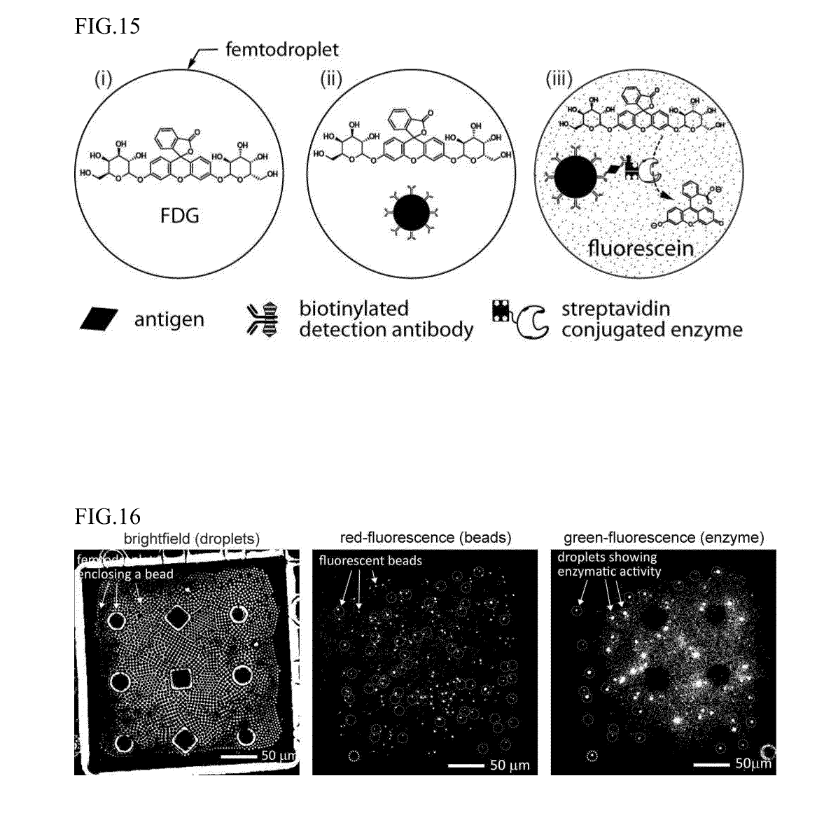

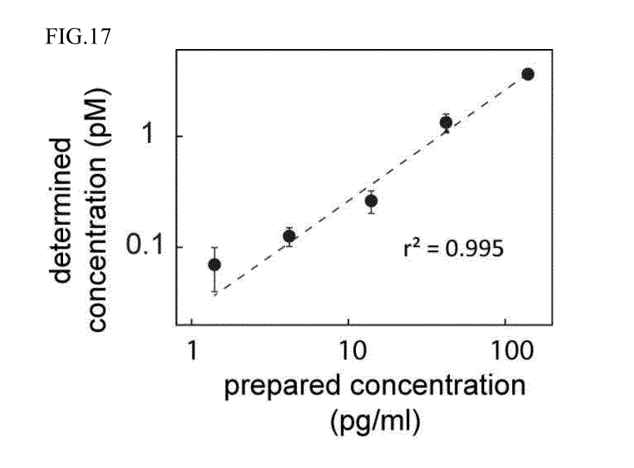

At the end of each experiment three different populations of femtodroplets were observed: i) droplets containing no bead; ii) droplets encapsulating a bead but without detectable enzymatic activity and iii) droplets containing a bead and a positive signal in green-fluorescence microscopy, corresponding to the presence of active enzyme conjugated to the target protein (FIG. 15). Since the concentration of PSA was lower than the bead concentration during anchoring of the target protein to the beads, Poisson statistics dictate that most beads capture either a single enzyme reporter or none. As the bead concentration was known, the fraction of bead-containing femtodroplets that exhibit enzymatic turnover to the total number of beads was used to calculate the concentration of PSA (FIG. 17). The linear relationship obtained between the known mass concentration and the experimentally-determined molar concentration confirmed that this approach can be used to quantify a low-abundance biomarker. Since the molar concentration in commercial PSA preparations is not known, the accuracy and precision of the assay was verified by comparing the molecular weight of PSA calculated from the experimental data (36.9±1.1 kDa) to the literature value (36 kDa). In the negative control—where the assay conditions were identical except that PSA was omitted—over 3,700 femtodroplets containing capture beads were analyzed, none of which exhibited detectable reporter fluorescence after incubation.

Another source of false positive signal would be free enzyme, not bound to beads. However, as femtodroplets enclosing a bead were specifically identified by their red fluorescence, those false positive signals were easily ruled out (FIG. 16). As a result, the lowest detectable analyte concentration was ultimately determined by the capacity of the current femtodroplet traps. As around 1,900 droplets encapsulating beads were analyzed per measurement, the theoretical limit of detection (i.e. the concentration required to generate an average of one fluorescent droplet in each experiment) is 5 fM.

1. A method for generation of microfluidic droplet made of a dispersion phase in a continuous phase with smaller than 500 fL in volume and more than 50000 droplets per second in generation rate, termed femtodroplets, the method comprising:
a step of ejecting a dispersion phase flowing in a plurality of dispersion phase-feeding microfluidic channels from a plurality of dispersion phase-feeding port toward a continuous phase flowing in a microfluidic channel in such a manner that flows of the dispersion phase and the continuous phase cross each other and part of the continuous phase extends through the dispersion phase-feeding port, whereby droplets are formed by the sheer force of the continuous phase;
a local constriction in depth and width of the channel wherein flows of the dispersion phase and the continuous phase cross each other, in which droplets are formed, is introduced within a section of the microfluidic channel.

2. A method as claimed in claim 1 wherein section of said local constriction spans less than 1500 μm, wherein depth of said constriction is less than 15 μm and wherein width of said constriction is less than 20 μm.

3. A method as claimed in claim 1 wherein section of said local constriction spans less than 300 μm, wherein depth of said constriction is less than 7 μm and wherein width of said constriction is less than 15 μm.

4. A method as claimed in claim 1 wherein the surface tension at the interface with said dispersion phase of said continuous phase is less than 50 (mN/m) and the viscosity of said continuous phase is less than 30 (cPs).

5. A method as claimed in claim 1 wherein the surface tension at the interface with said dispersion phase of said continuous phase is less than 5 (mN/m) and the viscosity of said continuous phase is less than 3 (cPs).

6. A method for storing said femtodroplets for duration of time, comprising:
a microfluidic component, the storage, integrated in said microfluidic device;
said storage made of a microfabricated elastomeric structure;
an elastomeric block formed with microfabricated processes, in which a portion of the elastomeric block is deflectable into one of the micro channel when the portion is actuated;
actuating said elastomeric block through introduced air or liquid pressure in said feeding port is less than 300 psi.
deflecting, sealing off said storage and dividing said storage into a number of traps by an actuation of said elastomeric blocks;
stopping and trapping a flow of a number of said femtodroplets within said traps in said storage;
the width of said traps is less than 1000 μm.

7. A method as claimed in claim 6 wherein a width of said elastomeric block is less than 2000 μm.

8. A method as claimed in claim 6 wherein a width of said elastomeric block is less than 300 μm.

9. A method as claimed in claim 6 wherein a width of trap is less than 3000 μm.

10. A method as claimed in claim 6 wherein a width of trap is less than 300 μm.

11. A method as claimed in claim 6 wherein said air or liquid pressure is less than 100 psi.

12. A method as claimed in claim 6 wherein depth of said storage component is less than 15 μm, wherein length of said storage component is less than 20 mm and wherein width of said storage component is less than 70 mm.

13. A method as claimed in claim 6 wherein depth of said storage component is less than 5 μm, wherein length of said storage component is less than 2 mm and wherein width of said storage component is less than 7 mm.

14. A method for determining a measure of the concentration of analyte molecules in a fluid sample, termed the femtodroplet immunoassay, the method comprising:
mixing a solution containing at least one type of analyte molecules with a number of capture particles that each include a binding surface having affinity for at least one type of analyte molecule;
immobilizing at least one type of analyte molecules on said capture particles such that said capture particles associate with at least one analyte molecule;
encapsulating at least a portion of said capture particles after the immobilizing step into said femtodroplets;
storing and keeping at least a portion of said femtodroplets after the encapsulation step in a plurality of said traps in a plurality of said storages in said microfluidic device;
interrogating a portion of said stored femtodroplets after the storing step and determining the number of said femtodroplets containing at least one analyte molecule;
determining a measure of the concentration of said analyte molecules in the fluid sample based at least in part on the number of said femtodroplets determined to contain at least one analyte molecule or particle;

15. The method as claimed in claim 14, wherein in the interrogation step, the number of said femtodroplets containing plurality of said capture particle containing at least one type of said analyte molecule or said capture particle not containing an analyte molecule is determined.

16. The method as claimed in claim 14, wherein the measure of the concentration of analyte molecule in the fluid sample is based at least in part on the ratio of the number of said femtodroplets interrogated in the interrogation step determined to contain said capture particle containing at least one analyte molecule, to the total number of said femtodroplets addressed in the interrogation step determined to contain a said capture particle.

17. The method as claimed in claim 14, wherein the plurality of capture particles that include a binding surface having affinity for at least one type of analyte molecule comprises a plurality of fluorescent, chromogenic or chemiluminescent beads.

18. The method as claimed in claim 14, wherein the average diameter of the plurality of capture particles is between about 0.05 micrometer and about the diameter of said femtodroplets.

19. The method as claimed in claim 14, wherein at least a portion of the analyte molecules are associated with at least one binding ligand, wherein the binding ligand comprises an enzymatic component.

20. The method of claim 14, wherein the binding surface comprises a plurality of capture components.
